SEQUENCE LISTING (22 sequences)

SEQ ID NO: 1 - protein, 2366 aa, Clostridium difficile
MSLVNRKQLEKMANVRFRVQEDEYVAILDALEEYHNMSENTVVEKYLKLKDINSLTDTYIDTYKKSGRNKALKKFKEYLVIEILELKNSNLTPVEKNLHFIWIGGQINDTAINYINQWKDVNSDYNVNVFYDSNAFLINTLKKTIIESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQELVERWNLAGASDILRVILKNIGGVYLDVDMLPGIHPDLFKDINKPDSVKTAVDWEEMQLEAIMKHKEYIPEYTSKHFDTLDEEVQSSFESVLASKSDKSEIFLPLGDIEVSPLEVKIAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFEEYKKNYFEGALGEDDNLDFSQNTVTDKEYLLEKISSSTKSSERGYVHYIVQLQGDKISYEAACNLFAKNPYDSILFQKNIEDSEVAYYYNPTDSEIQEIDKYRIPDRISDRPKIKLTFIGHGKAEFNTDIFAGLDVDSLSSEIETAIGLAKEDISPKSIEINLLGCNMFSYSVNVEETYPGKLLLRVKDKVSELMPSMSQDSIIVSANQYEVRINSEGRRELLDHSGEWINKEESIIKDISSKEYISFNPKENKIIVKSKNLPELSTLLQEIRNNSNSSDIELEEKVMLAECEINVISNIETQVVEERIEEAKSLTSDSINYIKNEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKVMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGSTNTSDSLMSFLESMNIKSIFVNFLQSNIKFILDANFIISGTTSIGQFEFICDENNNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFEDNYRGAVEWKELDGEMHYFSPETGKAFKGLNQIGDDKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFDPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 2 - protein, 543 aa, Clostridium difficile
MSLVNRKQLEKMANVRFRVQEDEYVAILDALEEYHNMSENTVVEKYLKLKDINSLTDTYIDTYKKSGRNKALKKFKEYLVIEILELKNSNLTPVEKNLHFIWIGGQINDTAINYINQWKDVNSDYNVNVFYDSNAFLINTLKKTIIESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQELVERWNLAGASDILRVAILKNIGGVYLDVDMLPGIHPDLFKDINKPDSVKTAVDWEEMQLEAIMKHKEYIPEYTSKHFDTLDEEVQSSFESVLASKSDKSEIFLPLGDIEVSPLEVKIAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFEEYKKNYFEGA

SEQ ID NO: 3 - protein, 381 aa, Clostridium difficile
LTTATTAIITSSLGIASGFSILLVPLAGISAGIPSLVNNELVLRDKATKVVDYFKHVSLVETEGVFTLLDDKVMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIG

SEQ ID NO: 4 - protein, 410 aa, Artificial Sequence
  TD1 Immunogen (aa 1072-1452) + peptide linker + 10x His Tag
  MISC_FEATURE (383)..(398): Peptide linker
  MISC_FEATURE (399)..(408): His tag
SLTTATTAIITSSLGIASGFSILLVPLAGISAGIPSLVNNELVLRDKATKVVDYFKHVSLVETEGVFTLLDDKVMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGEFSSGHIDDDDSHMLEHHHHHHHHHHGM

SEQ ID NO: 5 - protein, 421 aa, Clostridium difficile
TSDPLLRQEIEAKIGIMAVNLTTATTAIITSSLGIASGFSILLVPLAGISAGIPSLVNNELVLRDKATKVVDYFKHVSLVETEGVFTLLDDKVMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKE

SEQ ID NO: 6 - protein, 481 aa, Clostridium difficile
LLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKVMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDD

SEQ ID NO: 7 - protein, 533 aa, Clostridium difficile
MSLVNRKQLEKMANVRFRVQEDEYVAILDALEEYHNMSENTVVEKYLKLKDINSLTDTYIDTYKKSGRNKALKKFKEYLVIEILELKNSNLTPVEKNLHFIWIGGQINDTAINYINQWKDVNSDYNVNVFYDSNAFLINTLKKTIIESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQELVERWNLAGASDILRVAILKNIGGVYLDVDMLPGIHPDLFKDINKPDSVKTAVDWEEMQLEAIMKHKEYIPEYTSKHFDTLDEEVQSSFESVLASKSDKSEIFLPLGDIEVSPLEVKIAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFE

SEQ ID NO: 8 - protein, 593 aa, Clostridium difficile
MSLVNRKQLEKMANVRFRVQEDEYVAILDALEEYHNMSENTVVEKYLKLKDINSLTDTYIDTYKKSGRNKALKKFKEYLVIEILELKNSNLTPVEKNLHFIWIGGQINDTAINYINQWKDVNSDYNVNVFYDSNAFLINTLKKTIIESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQELVERWNLAGASDILRVAILKNIGGVYLDVDMLPGIHPDLFKDINKPDSVKTAVDWEEMQLEAIMKHKEYIPEYTSKHFDTLDEEVQSSFESVLASKSDKSEIFLPLGDIEVSPLEVKIAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFEEYKKNYFEGALGEDDNLDFSQNTVTDKEYLLEKISSSTKSSERGYVHYIVQLQGDKISYE

SEQ ID NO: 9 - protein, 573 aa, Clostridium difficile
MSLVNRKQLEKMANVRFRVQEDEYVAILDALEEYHNMSENTVVEKYLKLKDINSLTDTYIDTYKKSGRNKALKKFKEYLVIEILELKNSNLTPVEKNLHFIWIGGQINDTAINYINQWKDVNSDYNVNVFYDSNAFLINTLKKTIIESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQELVERWNLAGASDILRVAILKNIGGVYLDVDMLPGIHPDLFKDINKPDSVKTAVDWEEMQLEAIMKHKEYIPEYTSKHFDTLDEEVQSSFESVLASKSDKSEIFLPLGDIEVSPLEVKIAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFEEYKKNYFEGALGEDDNLDFSQNTVTDKEYLLEKISSSTKS

SEQ ID NO: 10 - protein, 254 aa, Clostridium difficile
PSLVNNELVLRDKATKVVDYFKHVSLVETEGVFTLLDDKVMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTI

SEQ ID NO: 11 - protein, 41 aa, Clostridium difficile
EYVAILDALEEYHNMSENTVVEKYLKLKDINSLTDTYIDTY

SEQ ID NO: 12 - protein, 392 aa, Clostridium difficile
ESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQELVERWNLAGASDILRVAILKNIGGVYLDVDMLPGIHPDLFKDINKPDSVKTAVDWEEMQLEAIMKHKEYIPEYTSKHFDTLDEEVQSSFESVLASKSDKSEIFLPLGDIEVSPLEVKIAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFEEYKKN

SEQ ID NO: 13 - protein, 52 aa, Clostridium difficile
PVSEIILSFTPSYYEDGLIGYDLGLVSLYNEKFYINNFGMMVSGLIYINDSL

SEQ ID NO: 14 - protein, 176 aa, Clostridium difficile
GLDVDSLSSEIETAIGLAKEDISPKSIEINLLGCNMFSYSVNVEETYPGKLLLRVKDKVSELMPSMSQDSIIVSANQYEVRINSEGRRELLDHSGEWINKEESIIKDISSKEYISFNPKENKIIVKSKNLPELSTLLQEIRNNSNSSDIELEEKVMLAECEINVISNIETQVVEER

SEQ ID NO: 15 - protein, 100 aa, Clostridium difficile
QYEVRINSEGRRELLDHSGEWINKEESIIKDISSKEYISFNPKENKIIVKSKNLPELSTLLQEIRNNSNSSDIELEEKVMLAECEINVISNIETQVVEER

SEQ ID NO: 16 - protein, 541 aa, Clostridium difficile
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYRANSLFTEQELLNIYSQELLNRGNLAAASDIVRLLALKNFGGVYLDVDMLPGIHSDLFKTDFINLQENTIEKTLKASDLIEFKFPENNLSQLTEQEINSLWSFDQASAKYQFEKYVRDYTGGS

SEQ ID NO: 17 - protein, 380 aa, Clostridium difficile
MSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLG

SEQ ID NO: 18 - protein, 41 aa, Clostridium difficile
EYKTILTNLDEYNKLTTNNNENKYLQLKKLNESIDVFMNKY

SEQ ID NO: 19 - protein, 390 aa, Clostridium difficile
SSTTEALQLLEEEIQNPQFDNMKFYKKRMEFIYDRQKRFINYYKSQINKPTVPTIDDIIKSHLVSEYNRDETVLESYRTNSLRKINSNHGIDIISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYRANSLFTEQELLNIYSQELLNRGNLAAASDIVRLLALKNFGGVYLDVDMLPGIHSDLFKTDFINLQENTIEKTLKASDLIEFKFPENNLSQLTEQEINSLWSFDQASAKYQFEKYVRD

SEQ ID NO: 20 - protein, 52 aa, Clostridium difficile
SLGYIMSNFKSFNSENELDRDHLGFKIIDNKTYYYDEDSKLVKGLININNSL

SEQ ID NO: 21 - protein, 279 aa, Clostridium difficile
NKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEK

SEQ ID NO: 22 - protein, 100 aa, Clostridium difficile
QYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEK

(19) United States
(12) Patent Application Publication
(10) Pub. No.: US 2022/0062401 A1
(43) Pub. Date: Mar. 3, 2022

(54) VACCINE COMPOSITIONS FOR CLOSTRIDIUM DIFFICILE

(71) Applicant: THE REGENTS OF THE UNIVERSITY OF CALIFORNIA, Oakland, CA (US)

(72) Inventors: Rongsheng Jin, Irvine, CA (US); Philip Felgner, Irvine, CA (US); Peng Chen, Irvine, CA (US)

(21) Appl. No.: 17/531,058

(22) Filed: Nov. 19, 2021

Related U.S. Application Data
(63) Continuation-in-part of application No. PCT/US20/34070, filed on May 21, 2020.
(60) Provisional application No. 62/851,040, filed on May 21, 2019.

(51) Int. Cl. A61K 39/08; C07K 14/33

(52) U.S. Cl. CPC A61K 39/08; C07K 14/33

(57) ABSTRACT

Methods and compositions for treating or preventing C. difficile infection (CDI) through TcdB or TcdA holotoxins. The compositions feature immunogens or binding agents, such as antibodies, nanobodies (VHHs), single-domain antibodies (sdAbs), etc., based on one or a combination of neutralizing epitopes of TcdB or TcdA. Where immunogens inhibit the conformational changes necessary for pore formation by TcdB at an endosomal pH. Additionally, immunogens inhibit the movement of the scissile bond into the CPD cleavage side and a proper orientation of GTD relative to CPD, thus inhibiting cleavage of the GTD, which is required to activate the toxin. The present invention also describes vaccines for treatment of CDI, e.g., vaccines that target TcdB or TcdA.

### CROSS-REFERENCES TO RELATED APPLICATIONS

This application is a continuation-in-part and claims benefit of PCT Application No. PCT/US2020/034070 filed May 21, 2020, which claims benefit of U.S. Provisional Application No. 62/851,040 filed May 21, 2019, the specification(s) of which is/are incorporated herein in their entirety by reference.

### STATEMENT REGARDING FEDERALLY SPONSORED RESEARCH OR DEVELOPMENT

This invention was made with government support under Grant No. R01A1125704 and R01A1139087 awarded by National Institutes of Health and Grant No. HDTRA1-16-C-0009 and HDTRA1-18-1-0035 awarded by DOD/DTRA. The government has certain rights in the invention.

### REFERENCE TO A SEQUENCE LISTING

Applicant asserts that the paper copy of the Sequence Listing is identical to the Sequence Listing in computer readable form found on the accompanying computer file, entitled UCI_19_16_PCT_CIP_Sequencing_Listing_ST25. The content of the sequence listing is incorporated herein by reference in its entirety.

### FIELD OF THE INVENTION

The present invention relates to neutralizing the holotoxin of Clostridium difficile, more particularly, to a therapeutic composition and method for treating Clostridium difficile infection.

### BACKGROUND OF THE INVENTION

Clostridium difficile is classified as one of the top three urgent antibiotic resistance threats by the Centers for Disease Control and Prevention (CDC), and C. difficile infection (CDI) has become the most common cause of antibiotic-associated diarrhea and gastroenteritis-associated death in developed countries. The pathology of CDI is primarily mediated by two homologous exotoxins, TcdA and TcdB, which target and disrupt the colonic epithelium, leading to diarrhea and colitis. While the relative roles of these two toxins in the pathogenesis of CDI are not completely understood, recent studies showed that TcdB is more virulent than TcdA and more important for inducing the host inflammatory and innate immune responses.

TcdA (˜308 kDa) and TcdB (˜270 kDa) contain four functional domains: an N-terminal glucosyltransferase domain (GTD), a cysteine protease domain (CPD), a central transmembrane delivery and receptor-binding domain (Delivery/RBD), and a C-terminal combined repetitive oligopeptides (CROPs) domain (FIG. 1A). It is widely accepted that the toxins bind to cell surface receptors via the Delivery/RBD and the CROPs and enter the cells through endocytosis. Acidification in the endosome triggers conformational changes in the toxins that prompt the Delivery/RBD to form a pore and deliver the GTD and the CPD across the endosomal membrane.

In the cytosol, the CPD is activated by eukaryotic-specific inositol hexakisphosphate (InsP6, also known as phytic acid) and subsequently undergoes autoproteolysis to release the GTD. The GTD then glucosylates small GTPases of the Rho family, including Rho, Rac, and CDCl42. Glucosylation of Rho proteins inhibits their functions, leading to alterations in the actin cytoskeleton, cell-rounding, and ultimately apoptotic cell death. Numerous structures have been reported for fragments of TcdA and TcdB, which have provided tremendous insights into the functions of these toxin domains. However, it remains unknown how individual domains interact within the supertertiary structure of the holotoxin, and how the holotoxin dynamically responds in a precise stepwise manner to the environmental and cellular cues, such as low pH and InsP6, which lead to intoxication.

An anti-TcdB neutralizing antibody (bezlotoxumab) was recently approved by the US Food and Drug Administration (FDA) as a prevention against recurrent infection, as up to 35% of CDI patients suffer a recurrence and many may require multiple rounds of treatments. However, this antibody is not indicated for the treatment of CDI, nor for the prevention of CDI.

### BRIEF SUMMARY OF THE INVENTION

It is the object of the present invention to provide a therapeutic composition and method that allows for the neutralization of a holotoxin (i.e. TcdB or TcdA) of Clostridium difficile, as specified in the independent claims. Embodiments of the invention are given in the dependent claims. Embodiments of the present invention can be freely combined with each other if they are not mutually exclusive.

The present invention describes a therapeutic composition that comprises of one or more isolated polypeptides that neutralizes the primary holotoxins (TcdB or TcdA) of C. difficile. In some embodiment the isolated polypeptides are comprised of a group that bind to the holotoxin and inhibit its toxicity (function) thereby neutralizing it.

Additionally, the present invention may feature a method of neutralizing the primary holotoxins (TcdB or TcdA) of C. difficile. In some embodiment the method comprises producing an immunogen of a holotoxin (TcdB or TcdA) of C. difficile and introducing the immunogen to a host to elicit an immune response to the immunogen. In another embodiment the host produces an antibody specific for the holotoxin base on the immunogen.

Furthermore, the present invention may feature a method of designing and producing a vaccine specific for a holotoxin (TcdB or TcdA) of C. difficile.

Without wishing to limit the present invention to any theory or mechanism, it is believed that the vaccines of the present invention may be advantageous (e.g., compared to a toxoid vaccine for CDI) because the immunogens of the present invention are nontoxic, making them potentially safer; the immunogens of the present invention may be produced in E. coli with high yield and high purity, making them less expensive to produce, formulate, and store (production of vaccines can be challenging); the immunogens of the present invention keep their native 3D structure (as compared to the disrupted antigenic structures in a toxoid), and thus may be more efficient for triggering an immune response as a vaccine; and the immunogens of the present invention are small and contain known neutralizing epitopes, thus the immunogens may be more efficient for triggering the production of neutralizing antibodies. Further, because these immunogens are directed to a smaller (as compared to the whole holotoxin), more specific region of TcdB, it may result in a better immune response. The present invention provides polypeptides that are smaller than the whole holotoxin but larger than small (e.g., 15-mer) peptides: mid-sized peptides that have well-defined 3D structure.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A shows a schematic diagram of the full length TcdB holotoxin, showing the domain organization of TcdB: GTD (red); CPD (purple); Delivery/RBD (yellow); CROPs (blue) and the approximate VHH-binding regions.

FIG. 1B shows a schematic representation of the 3D structure of TcdB holotoxin. The 3 VHHs that were used to facilitate crystallization were omitted for clarity (GTD; CPD; Delivery/RBD; CROPs).

FIG. 2A shows a schematic diagram of the CROPs domain showing the organization of the short repeats (SRs, thin blue bars) and the long repeats (LRs, thick black bars). The dashed lines indicate the boundaries of four CROPs units (I-IV).

FIG. 2B shows a close-up view into the CROPs domain while the rest of TcdB is in a surface representation.

FIG. 2C shows the superposition of the 4 CROPs units. The LR in each CROPs unit causes a ˜132-146° kink.

FIG. 2D and FIG. 2E show the hinge region, which connects the CROPs domain to the rest of the toxin, is located at the center of the TcdB and surrounded by the GTD, the CPD, and the Delivery/RBD.

FIG. 3A shows a curve-fit analysis in SAXS studies, showing that the CROPs domain undergoes pH-dependent conformational changes. The theoretical Kratky plot based on the structure of TcdB holotoxin is nearly identical to the experimental scattering profile at pH 5.0 (upper panel), but different from that at pH 7.4 (lower panel).

FIG. 3B shows cross-linked peptides between different TcdB domains identified by XL-MS.

FIG. 3C shows XL-MS results, suggesting that TcdB could adopt a “closed” conformation at neutral pH, where the central portion and the C-terminal tip of the CROPs domain move within ˜30 Å of the Delivery/RBD.

FIG. 3D shows a model of the two limiting structure states of TcdB holotoxin. The acceptor dye on the GTD-bound 7F and the donor dye (hexagon) on the CROPs domain-bound B39 (star) are shown.

FIG. 3E shows a population histogram of unaveraged FRET efficiency from TcdB in complex with dye-labeled VHHs at pH 5.0 (n=498) and pH 7.0 (n=594).

FIG. 4A shows a pore-forming intermediate state of TcdB. 5D binds to the Delivery/RBD and directly interacts with the pore-forming region. The pore-forming region is shown in a ribbon model while the rest of the toxin is shown in a surface model.

FIG. 4B shows a representative 2Fo-Fc electron density map of a portion of the pore-forming region contoured at 1.0 σ, which was overlaid with the final refined model.

FIG. 4C shows the amino acid sequence alignment of the pore-forming region among different members in the large clostridial glucosylating toxins (LCGT) family. TcdB*, TcdB, and TcdB2 are produced by the M68 strain, the VPI 10463 strain, and the BI/NAP1/027 strain, respectively. Secondary structures of TcdB* and TcdA are shown on the top and the bottom, respectively. Residues 1032-1047 in TcdB holotoxin that have no visible electron density are indicated by “x”.

FIG. 4D shows that TcdB at acidic pH (purple) and TcdA at neutral pH (orange) adopt drastically different conformations in the pore-forming region. The two structures were superimposed based on the Delivery/RBD.

FIG. 4E shows a calcein dye release assay. TcdB (0-25 nM) was tested with liposomes loaded with 50 mM calcein at pH 4.6, in the presence or absence of 5D or 7F.

FIG. 4F shows a membrane depolarization assay. Liposomes were polarized at a positive internal voltage by adding valinomycin in the presence of a transmembrane KCl gradient. Membrane potential was measured using the voltage-sensitive fluorescence dye ANS (8-anilinonaphthalene-1-sulfonic acid). After 3 min, TcdB with various concentrations of 5D or 7F was added. Data presented as mean±SEM, n=3.

FIG. 5A shows a schematic diagram showing the locations of the β-flap, the 3 helical bundle (3-HB), and the hinge in the primary sequence of TcdB.

FIG. 5B shows the superposition of the apo CPD (grey coils) in TcdB holotoxin and a CPD fragment bound with InsP6. The zinc atom in the apo CPD is shown as a sphere, and InsP6 is in a stick model.

FIG. 5C and FIG. 5D show the β-flap, the 3-HB, and the hinge co-localize at the center of TcdB.

FIG. 6 shows antibody titers of various nanobead subunit vaccines.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

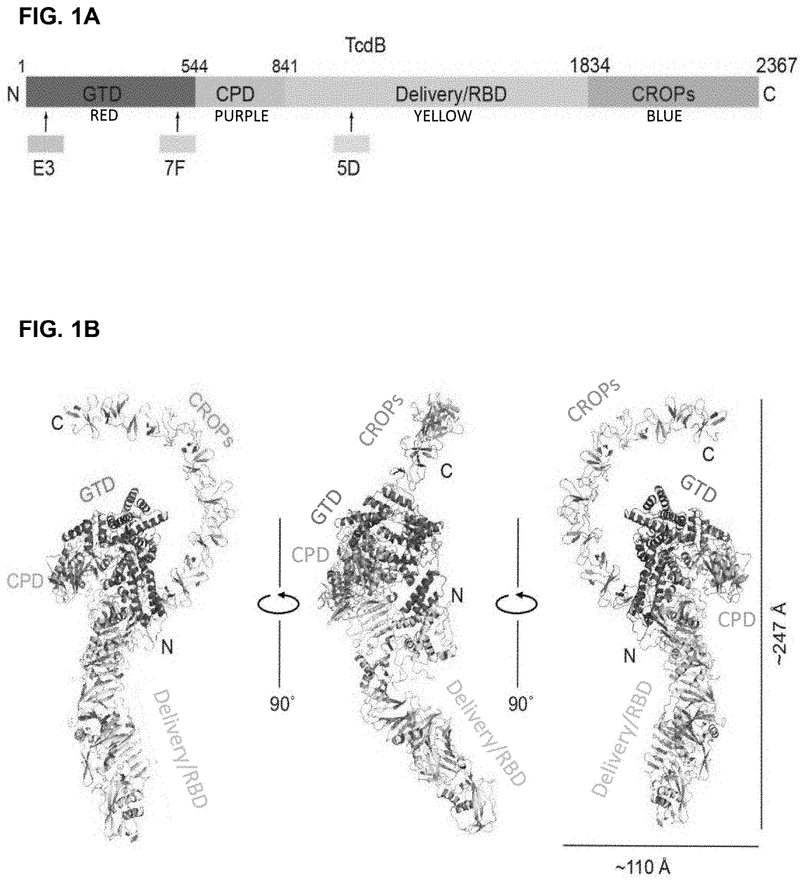

Referring now to FIGS. 1-6, the present invention features a therapeutic composition and method for neutralizing the primary holotoxin (i.e. TcdB and TcdA) of C. difficile to potentially treat and prevent CDI. Additionally, the present invention features a method of producing a vaccine for a holotoxin of C.

As used herein, the sequence of TcdB from C. difficile is from the M68 strain (WP_003426838.1, see Table 1 below). All amino acid numberings are in reference to this sequence.

TABLE 1

| Description: | Sequence: |
|---|---|
| TcdB of C. | MSLVNRKQLEKMANVRFRVQEDEYVAILDALEEYHNMSENTVVEKYLKLKDINSLTDT |

difficile

| | |
|---|---|
| M68 strain | YIDTYKKSGRNKALKKFKEYLVIEILELKNSNLTPVEKNLHFIWIGGQINDTAINYINQWK |

(SEQ ID NO:

| | |
|---|---|
| 1) | DVNSDYNVNVFYDSNAFLINTLKKTIIESASNDTLESFRENLNDPEFNHTAFFRKRMQIIY |
| | DKQQNFINYYKAQKEENPDLIIDDIVKTYLSNEYSKDIDELNAYIEESLNKVTENSGNDVR |
| | NFEEFKTGEVFNLYEQELVERWNLAGASDILRVILKNIGGVYLDVDMLPGIHPDLFKDIN |
| | KPDSVKTAVDWEEMQLEAIMKHKEYIPEYTSKHFDTLDEEVQSSFESVLASKSDKSEIFLP |
| | LGDIEVSPLEVKIAFAKGSIINQALISAKDSYCSDLLIKQIQNRYKILNDTLGPIISQGNDFNT |
| | TMNNFGESLGAIANEENISFIAKIGSYLRVGFYPEANTTITLSGPTIYAGAYKDLLTFKEMS |
| | IDTSILSSELRNFEFPKVNISQATEQEKNSLWQFNEERAKIQFEEYKKNYFEGALGEDDNL |
| | DFSQNTVTDKEYLLEKISSSTKSSERGYVHYIVQLQGDKISYEAACNLFAKNPYDSILFQK |
| | NIEDSEVAYYYNPTDSEIQEIDKYRIPDRISDRPKIKLTFIGHGKAEFNTDIFAGLDVDSLSS |
| | EIETAIGLAKEDISPKSIEINLLGCNMFSYSVNVEETYPGKLLLRVKDKVSELMPSMSQDSI |
| | IVSANQYEVRINSEGRRELLDHSGEWINKEESIIKDISSKEYISFNPKENKIIVKSKNLPELST |
| | LLQEIRNNSNSSDIELEEKVMLAECEINVISNIETQVVEERIEEAKSLTSDSINYIKNEFKLIE |
| | SISDALCDLKQQNELEDSHFISFEDISETDEGFSIRFINKETGESIFVETEKTIFSEYANHITEE |
| | ISKIKGTIFDTVNGKLVKKVNLDTTHEVNTLNAAFFIQSLIEYNSSKESLSNLSVAMKVQV |
| | YAQLFSTGLNTITDAAKVVELVSTALDETIDLLPTLSEGLPIIATIIDGVSLGAAIKELSETS |
| | DPLLRQEIEAKIGIMAVNLTTATTAIITSSLGIASGFSILLVPLAGISAGIPSLVNNELVLRDK |
| | ATKVVDYFKHVSLVETEGVFTLLDDKVMMPQDDLVISEIDFNNNSIVLGKCEIWRMEGG |
| | SGHTVTDDIDHFFSAPSITYREPHLSIYDVLEVQKEELDLSKDLMVLPNAPNRVFAWETG |
| | WTPGLRSLENDGTKLLDRIRDNYEGEFYWRYFAFIADALITTLKPRYEDTNIRINLDSNTR |
| | SFIVPIITTEYIREKLSYSFYGSGGTYALSLSQYNMGINIELSESDVWIIDVDNVVRDVTIES |
| | DKIKKGDLIEGILSTLSIEENKIILNSHEINFSGEVNGSNGFVSLTFSILEGINAIIEVDLLSKS |
| | YKLLISGELKILMLNSNHIQQKIDYIGFNSELQKNIPYSFVDSEGKENGFINGSTKEGLFVS |
| | ELPDVVLISKVYMDDSKPSFGYYSNNLKDVKVITKDNVNILTGYYLKDDIKISLSLTLQDE |
| | KTIKLNSVHLDESGVAEILKFMNRKGSTNTSDSLMSFLESMNIKSIFVNFLQSNIKFILDAN |
| | FIISGTTSIGQFEFICDENNNIQPYFIKFNTLETNYTLYVGNRQNMIVEPNYDLDDSGDISST |
| | VINFSQKYLYGIDSCVNKVVISPNIYTDEINITPVYETNNTYPEVIVLDANYINEKINVNIND |
| | LSIRYVWSNDGNDFILMSTSEENKVSQVKIRFVNVFKDKTLANKLSFNFSDKQDVPVSEII |
| | LSFTPSYYEDGLIGYDLGLVSLYNEKFYINNFGMMVSGLIYINDSLYYFKPPVNNLITGFV |
| | TVGDDKYYFNPINGGAASIGETIIDDKNYYFNQSGVLQTGVFSTEDGFKYFAPANTLDEN |
| | LEGEAIDFTGKLIIDENIYYFEDNYRGAVEWKELDGEMHYFSPETGKAFKGLNQIGDDKY |
| | YFNSDGVMQKGFVSINDNKHYFDDSGVMKVGYTEIDGKHFYFAENGEMQIGVFNTEDG |
| | FKYFAHHNEDLGNEEGEEISYSGILNFNNKIYYFDDSFTAVVGWKDLEDGSKYYFDEDT |
| | AEAYIGLSLINDGQYYFNDDGIMQVGFVTINDKVFYFSDSGIIESGVQNIDDNYFYIDDNG |
| | IVQIGVFDTSDGYKYFAPANTVNDNIYGQAVEYSGLVRVGEDVYYFGETYTIETGWIYD |
| | MENESDKYYFDPETKKACKGINL1DDIKYYFDEKGIMRTGLISFENNNYYFNENGEMQFG |
| | YINIEDKMFYFGEDGVMQIGVFNTPDGFKYFAHQNTLDENFEGESINYTGWLDLDEKRY |
| | YFTDEYIAATGSVIIDGEEYYFDPDTAQLVISE |

Referring to Table 1 and FIG. 1A, the TcdB holotoxin has an N-terminal glucosyltransferase domain (GTD) from amino acids 1-544, a cysteine protease domain (CPD) from amino acids 545-841, a delivery domain/receptor binding domain (Delivery/RBD) from amino acids 842 to 1834, and a C-terminal combined repetitive oligopeptides (CROPs) domain from amino acids 1835 to 2367. Additionally, there are three neutralizing epitopes: E3 (in the GTD, encompassing amino acids 23-63); 7F (C-terminus of the GTD immediately juxtaposed to the cleavage site, encompassing amino acids 147-538), and 5D (a portion of the Delivery/RBD, encompassing amino acids 1105-1358). Although the regions encompassed by the neutralizing epitopes are not linear in the primary amino acid sequence, they do cluster together in 3D forming the epitope.

In some embodiment, the present invention features a therapeutic composition that comprises of one or more isolated polypeptides that neutralizes the primary holotoxins of C. difficile. In some embodiment, the isolated polypeptide comprises a sequence that binds the holotoxin and inhibits toxicity/function thereby neutralizing it. In some embodiment the polypeptide sequence may be used as an immunogen or targets for binding agents or other drugs.

Various immunogens for C. difficile TcdB were produced (see Table 2): TcdB-FL (full length TcdB); GTD (aa 1-543, SEQ ID NO: 2), TD (aa 798-1805, sequence not shown); TD3 (aa 1286-1805, sequence not shown); CROP4 (aa 2235-2367, sequence not shown); and TD1 (aa 1072-1452, the pore-B epitope, SEQ ID NO: 3). TD refers to translocation domain.

Table 2 below describes non-limiting examples of polypeptide sequences that may be used as immunogens or as targets for binding agents or other drugs.

TABLE 2

| | | SEQ |
|---|---|---|
| | | ID |
| Description | Sequence | NO: |
| GTD aa 1-543 | MSLVNRKQLEKMANVRFRVQEDEYVAILDALEEYHNMSENTVVEKYLKLKDI | 2 |
| | NSLTDTYIDTYKKSGRNKALKKFKEYLVIEILELKNSNLTPVEKNLHFIWIGGQI | |
| | NDTAINYINQWKDVNSDYNVNVFYDSNAFLINTLKKTIIESASNDTLESFRENL | |
| | NDPEFNHTAFFRKRMQIIYDKQQNFINYYKAQKEENPDLIIDDIVKTYLSNEYSK | |
| | DIDELNAYIEESLNKVTENSGNDVRNFEEFKTGEVFNLYEQELVERWNLAGAS | |
| | DILRVAILKNIGGVYLDVDMLPGIHPDLFKDINKPDSVKTAVDWEEMQLEAIM | |
| | KHKEYIPEYTSKHFDTLDEEVQSSFESVLASKSDKSEIFLPLGDIEVSPLEVKIAF | |
| | AKGSIINQALISAKDSYCSDLLIKQIQNRYKILNDTLGPIISQGNDFNTTMNNFGE | |
| | SLGAIANEENISFIAKIGSYLRVGFYPEANTTITLSGPTIYAGAYKDLLTFKEMSID | |
| | TSILSSELRNFEFPKVNISQATEQEKNSLWQFNEERAKIQFEEYKKNYFEGA | |
| TD1 aa | LTTATTAIITSSLGIASGFSILLVPLAGISAGIPSLVNNELVLRDKATKVVDYFKH | 3 |
| 1072-1452 | VSLVETEGVFTLLDDKVMMPQDDLVISEIDFNNNSIVLGKCEIWRMEGGSGHT | |
| | VTDDIDHFFSAPSITYREPHLSIYDVLEVQKEELDLSKDLMVLPNAPNRVFAWE | |
| | TGWTPGLRSLENDGTKLLDRIRDNYEGEFYWRYFAFIADALITTLKPRYEDTNI | |
| | RINLDSNTRSFIVPIITTEYIREKLSYSFYGSGGTYALSLSQYNMGINIELSESDVW | |
| | IIDVDNVVRDVTIESDKIKKGDLIEGILSTLSIEENKIILNSHEINFSGEVNGSNGFV | |
| | SLTFSILEGINAIIEVDLLSKSYKLLISGELKILMLNSNHIQQKIDYIG | |
| TD1 | SLTTATTAIITSSLGIASGFSILLVPLAGISAGIPSLVNNELVLRDKATKVVDYFKH | 4 |
| Immunogen | VSLVETEGVFTLLDDKVMMPQDDLVISEIDFNNNSIVLGKCEIWRMEGGSGHT | |
| (aa 1072- | VTDDIDHFFSAPSITYREPHLSIYDVLEVQKEELDLSKDLMVLPNAPNRVFAWE | |
| 1452 + peptide | TGWTPGLRSLENDGTKLLDRIRDNYEGEFYWRYFAFIADALITTLKPRYEDTNI | |
| linker | RINLDSNTRSFIVPIITTEYIREKLSYSFYGSGGTYALSLSQYNMGINIELSESDVW | |
| (under- | IIDVDNVVRDVTIESDKIKKGDLIEGILSTLSIEENKIILNSHEINFSGEVNGSNGFV | |
| lined) + | SLTFSILEGINAIIEVDLLSKSYKLLISGELKILMLNSNHIQQKIDYIGEFSSGHIDD | |
| 10× His | DDSHMLEHHHHHHHHHHGM | |
| Tag (bold) | | |
| aa 1052-1472 | TSDPLLRQEIEAKIGIMAVNLTTATTAIITSSLGIASGFSILLVPLAGISAGIPSLVN | 5 |
| of TcdB | NELVLRDKATKVVDYFKHVSLVETEGVFTLLDDKVMMPQDDLVISEIDFNNNS | |
| | IVLGKCEIWRMEGGSGHTVTDDIDHFFSAPSITYREPHLSIYDVLEVQKEELDLS | |
| | KDLMVLPNAPNRVFAWETGWTPGLRSLENDGTKLLDRIRDNYEGEFYWRYFA | |
| | FIADALITTLKPRYEDTNIRINLDSNTRSFIVPIITTEYIREKLSYSFYGSGGTYALS | |
| | LSQYNMGINIELSESDVWIIDVDNVVRDVTIESDKIKKGDLIEGILSTLSIEENKII | |
| | LNSHEINFSGEVNGSNGFVSLTFSILEGINAIIEVDLLSKSYKLLISGELKILMLNS | |
| | NHIQQKIDYIGFNSELQKNIPYSFVDSEGKE | |
| aa 1022-1502 | LLPTLSEGLPIIATIIDGVSLGAAIKELSETSDPLLRQEIEAKIGIMAVNLTTATTAII | 6 |
| of TcdB | TSSLGIASGFSILLVPLAGISAGIPSLVNNELVLRDKATKVVDYFKHVSLVETEG | |
| | VFTLLDDKVMMPQDDLVISEIDFNNNSIVLGKCEIWRMEGGSGHTVTDDIDHFF | |
| | SAPSITYREPHLSIYDVLEVQKEELDLSKDLMVLPNAPNRVFAWETGWTPGLRS | |
| | LENDGTKLLDRIRDNYEGEFYWRYFAFIADALITTLKPRYEDTNIRINLDSNTRS | |
| | FIVPIITTEYIREKLSYSFYGSGGTYALSLSQYNMGINIELSESDVWIIDVDNVVR | |
| | DVTIESDKIKKGDLIEGILSTLSIEENKIILNSHEINFSGEVNGSNGFVSLTFSILEGI | |
| | NAIIEVDLLSKSYKLLISGELKILMLNSNHIQQKIDYIGFNSELQKNIPYSFVDSEG | |
| | KENGFINGSTKEGLFVSELPDVVLISKVYMDD | |
| aa 1-533 | MSLVNRKQLEKMANVRFRVQEDEYVAILDALEEYHNMSENTVVEKYLKLKDI | 7 |
| of TcdB | NSLTDTYIDTYKKSGRNKALKKFKEYLVIEILELKNSNLTPVEKNLHFIWIGGQI | |
| | NDTAINYINQWKDVNSDYNVNVFYDSNAFLINTLKKTIIESASNDTLESFRENL | |
| | NDPEFNHTAFFRKRMQIIYDKQQNFINYYKAQKEENPDLIIDDIVKTYLSNEYSK | |
| | DIDELNAYIEESLNKVTENSGNDVRNFEEFKTGEVFNLYEQELVERWNLAGAS | |
| | DILRVAILKNIGGVYLDVDMLPGIHPDLFKDINKPDSVKTAVDWEEMQLEAIM | |
| | KHKEYIPEYTSKHFDTLDEEVQSSFESVLASKSDKSEIFLPLGDIEVSPLEVKIAF | |
| | AKGSIINQALISAKDSYCSDLLIKQIQNRYKILNDTLGPIISQGNDFNTTMNNFGE | |
| | SLGAIANEENISFIAKIGSYLRVGFYPEANTTITLSGPTIYAGAYKDLLTFKEMSID | |
| | TSILSSELRNFEFPKVNISQATEQEKNSLWQFNEERAKIQFE | |
| aa 1-593 | MSLVNRKQLEKMANVRFRVQEDEYVAILDALEEYHNMSENTVVEKYLKLKDI | 8 |
| of TcdB | NSLTDTYIDTYKKSGRNKALKKFKEYLVIEILELKNSNLTPVEKNLHFIWIGGQI | |
| | NDTAINYINQWKDVNSDYNVNVFYDSNAFLINTLKKTIIESASNDTLESFRENL | |
| | NDPEFNHTAFFRKRMQIIYDKQQNFINYYKAQKEENPDLIIDDIVKTYLSNEYSK | |
| | DIDELNAYIEESLNKVTENSGNDVRNFEEFKTGEVFNLYEQELVERWNLAGAS | |
| | DILRVAILKNIGGVYLDVDMLPGIHPDLFKDINKPDSVKTAVDWEEMQLEAIM | |
| | KHKEYIPEYTSKHFDTLDEEVQSSFESVLASKSDKSEIFLPLGDIEVSPLEVKIAF | |
| | AKGSIINQALISAKDSYCSDLLIKQIQNRYKILNDTLGPIISQGNDFNTTMNNFGE | |
| | SLGAIANEENISFIAKIGSYLRVGFYPEANTTITLSGPTIYAGAYKDLLTFKEMSID | |
| | TSILSSELRNFEFPKVNISQATEQEKNSLWQFNEERAKIQFEEYKKNYFEGALGE | |
| | DDNLDFSQNTVTDKEYLLEKISSSTKSSERGYVHYIVQLQGDKISYE | |
| aa 1-573 | MSLVNRKQLEKMANVRFRVQEDEYVAILDALEEYHNMSENTVVEKYLKLKDI | 9 |
| of TcdB | NSLTDTYIDTYKKSGRNKALKKFKEYLVIEILELKNSNLTPVEKNLHFIWIGGQI | |
| | NDTAINYINQWKDVNSDYNVNVFYDSNAFLINTLKKTIIESASNDTLESFRENL | |
| | NDPEFNHTAFFRKRMQIIYDKQQNFINYYKAQKEENPDLIIDDIVKTYLSNEYSK | |
| | DIDELNAYIEESLNKVTENSGNDVRNFEEFKTGEVFNLYEQELVERWNLAGAS | |
| | DILRVAILKNIGGVYLDVDMLPGIHPDLFKDINKPDSVKTAVDWEEMQLEAIM | |
| | KHKEYIPEYTSKHFDTLDEEVQSSFESVLASKSDKSEIFLPLGDIEVSPLEVKIAF | |
| | AKGSIINQALISAKDSYCSDLLIKQIQNRYKILNDTLGPIISQGNDFNTTMNNFGE | |
| | SLGAIANEENISFIAKIGSYLRVGFYPEANTTITLSGPTIYAGAYKDLLTFKEMSID | |
| | TSILSSELRNFEFPKVNISQATEQEKNSLWQFNEERAKIQFEEYKKNYFEGALGE | |
| | DDNLDFSQNTVTDKEYLLEKISSSTKS | |
| aa 1105- | PSLVNNELVLRDKATKVVDYFKHVSLVETEGVFTLLDDKVMMPQDDLVISEID | 10 |
| 1358 of | FNNNSIVLGKCEIWRMEGGSGHTVTDDIDHFFSAPSITYREPHLSIYDVLEVQKE | |
| TcdB (5D) | ELDLSKDLMVLPNAPNRVFAWETGWTPGLRSLENDGTKLLDRIRDNYEGEFY | |
| | WRYFAFIADALITTLKPRYEDTNIRINLDSNTRSFIVPIITTEYIREKLSYSFYGSG | |
| | GTYALSLSQYNMGINIELSESDVWIIDVDNVVRDVTI | |
| aa 23-63 of | EYVAILDALEEYHNMSENTVVEKYLKLKDINSLTDTYIDTY | 11 |
| TcdB (E3) | | |
| aa 147-538 of | ESASNDTLESFRENLNDPEFNHTAFFRKRMQIIYDKQQNFINYYKAQKEENPDLI | 12 |
| TcdB (F7) | IDDIVKTYLSNEYSKDIDELNAYIEESLN | |
| | KVTENSGNDVRNFEEFKTGEVFNLYEQELVERWNLAGADILRVAILKNIGGVY | |
| | LDVDMLPGIHPDLFKDINKPDSVKTAVDWEEMQLEAIMKHKEYIPEYTSKHFD | |
| | TLDEEVQSSEESVLASKSDKSEIFLPLGDIEVSPLEVKIAFAKGSIINQALISAKDS | |
| | YCSDLLIKQIQNRYKILNDTLGPIISQGNDFNTTMNNFGESLGAIANEENISFIAKI | |
| | GSYLRVGFYPEANTTITLSGPTIYAGAYKDLLTFKEMSIDTSILSSELRNFEFPKV | |
| | NISQATEQEKNSLWQFNEERAKIQFEEYKKN | |
| aa 1792- | PVSEIILSFTPSYYEDGLIGYDLGLVSLYNEKFYINNFGMMVSGLIYINDSL | 13 |
| 1843 of TcdB, | | |
| (hinge region) | | |
| aa 666-841 | GLDVDSLSSEIETAIGLAKEDISPKSIEINLLGCNMFSYSVNVEETYPGKLLLRVK | 14 |
| of TcdB, | DKVSELMPSMSQDSIIVSANQYEVRINSEGRRELLDHSGEWINKEESIIKDISSKE | |
| (3-HB) | YISFNPKENKIIVKSKNLPELSTLLQEIRNNSNSSDIELEEKVMLAECEINVISNIET | |
| | QVVEER | |
| aa 742-841 | QYEVRINSEGRRELLDHSGEWINKEESIIKDISSKEYISFNPKENKIIVKSKNLPEL | 15 |
| of TcdB, | STLLQEIRNNSNSSDIELEEKVMLAECEINVISNIETQVVEER | |
| (β-flap) | | |
| aa 1-541 | MSLISKEELIKLAYSIRPRENEYKTILTNLDEYNKLTTNNNENKYLQLKKLNESI | 16 |
| of TcdA | DVFMNKYKTSSRNRALSNLKKDILKEVILIKNSNTSPVEKNLHFVWIGGEVSDI | |
| | ALEYIKQWADINAEYNIKLWYDSEAFLVNTLKKAIVESSTTEALQLLEEEIQNP | |
| | QFDNMKFYKKRMEFIYDRQKRFINYYKSQINKPTVPTIDDIIKSHLVSEYNRDET | |
| | VLESYRTNSLRKINSNHGIDIISRPSSIGLDRWEMIKLEAIMKYKKYINNYTSENF | |
| | DKLDQQLKDNFKLIIESKSEKSEIFSKLENLNVSDLEIKIAFALGSVINQALISKQG | |
| | SYLTNLVIEQVKNRYQFLNQHLNPAIESDNNFTDTTKIFHDSLFNSATAENSMFL | |
| | TKIAPYLQVGFMPEARSTISLSGPGAYASAYYRANSLFTEQELLNIYSQELLNRG | |
| | NLAAASDIVRLLALKNFGGVYLDVDMLPGIHSDLFKTDFINLQENTIEKTLKAS | |
| | DLIEFKFPENNLSQLTEQEINSLWSFDQASAKYQFEKYVRDYTGGS | |
| aa 1073-1452 | MSLSIAATVASIVGIGAEVTIFLLPIAGISAGIPSLVNNELILHDKATSVVNYFNHL | 17 |
| of TcdA | SESKKYGPLKTEDDKILVPIDDLVISEIDFNNNSIKLGTCNILAMEGGSGHTVTG | |
| | NIDHFFSSPSISSHIPSLSIYSAIGIETENLDFSKKIMMLPNAPSRVFWWETGAVPG | |
| | LRSLENDGTRLLDSIRDLYPGKFYWRFYAFFDYAITTLKPVYEDTNIKIKLDKDT | |
| | RNFIMPTITTNEIRNKLSYSFDGAGGTYSLLLSSYPISTNINLSKDDLWIFNIDNEV | |
| | REISIENGTIKKGKLIKDVLSKIDINKNKLIIGNQTIDFSGDIDNKDRYIFLTCELD | |
| | DKISLIIEINLVAKSYSLLLSGDKNYLISNLSNTIEKINTLG | |
| aa 22-62 | EYKTILTNLDEYNKLTTNNNENKYLQLKKLNESIDVFMNKY | 18 |
| of TcdA | | |
| aa 146-536 | SSTTEALQLLEEEIQNPQFDNMKFYKKRMEFIYDRQKRFINYYKSQINKPTVPTI | 19 |
| of TcdA | DDIIKSHLVSEYNRDETVLESYRTNSLRKINSNHGIDIISRPSSIGLDRWEMIKLEA | |
| | IMKYKKYINNYTSENFDKLDQQLKDNFKLIIESKSEKSEIFSKLENLNVSDLEIKI | |
| | AFALGSVINQALISKQGSYLTNLVIEQVKNRYQFLNQHLNPAIESDNNFTDTTKI | |
| | FHDSLFNSATAENSMFLTKIAPYLQVGFMPEARSTISLSGPGAYASAYYRANSL | |
| | FTEQELLNIYSQELLNRGNLAAASDIVRLLALKNFGGVYLDVDMLPGIHSDLFK | |
| | TDFINLQENTIEKTLKASDLIEFKFPENNLSQLTEQEINSLWSFDQASAKYQFEK | |
| | YVRD | |
| aa 1789-1840 | SLGYIMSNFKSFNSENELDRDHLGFKIIDNKTYYYDEDSKLVKGLININNSL | 20 |
| of TcdA | | |
| aa 664-842 | NKIPSNNVEEAGSKNYVHYIIQLQGDDISYEATCNLFSKNPKNSIIIQRNMNESA | 21 |
| of TcdA | KSYFLSDDGESILELNKYRIPERLKNKEKVKVTFIGHGKDEFNTSEFARLSVDSL | |
| | SNEISSFLDTIKLDISPKNVEVNLLGCNMFSYDFNVEETYPGKLLLSIMDKITSTL | |
| | PDVNKNSITIGANQYEVRINSEGRKELLAHSGKWINKEEAIMSDLSSKEYIFFDSI | |
| | DNKLKAKSKNIPGLASISEDIKTLLLDASVSPDTKFILNNLKLNIESSIGDYIYYEK | |
| aa 743-842 | QYEVRINSEGRKELLAHSGKWINKEEAIMSDLSSKEYIFFDSIDNKLKAKSKNIP | 22 |
| of TcdA | GLASISEDIKTLLLDASVSPDTKFILNNLKLNIESSIGDYIYYEK | |

Now referring to Table 2, the present invention features isolated polypeptides, not limited to the sequences listed here. SEQ ID NO: 2, refers to amino acids 1-543 of TcdB of C. difficile. SEQ ID NO: 3, refers to amino acids 1072-1452 of TcdB of C. difficile and amino acids 1072-1452 are a portion of a translocation domain necessary for pore formation. SEQ ID NO: 5, refers to amino acids 1052-1472 of TcdB of C. difficile. SEQ ID NO: 6, refers to amino acids 1022-1502 of TcdB of C. difficile. SEQ ID NO: 7, refers to amino acids 1-533 of TcdB of C. difficile. SEQ ID NO: 8, refers to amino acids 1-593 of TcdB of C. difficile. SEQ ID NO: 9, refers to amino acids 1-573 of TcdB of C. difficile. SEQ ID NO: 10, refers to amino acids 1105-1358 of TcdB of C. difficile, and is the region that encompasses the 5D epitope. SEQ ID NO: 11, refers to amino acids 23-63 of TcdB of C. difficile and is the region that encompasses the E3 epitope. SEQ ID NO: 12, refers to amino acids 147-538 of TcdB of C. difficile and encompasses the F7 epitope. SEQ ID NO: 13, refers to amino acids 1792-1845 of TcdB of C. difficile which corresponds to the hinge region. SEQ ID NO: 14, refers to amino acids 666-841 of TcdB of C. difficile which corresponds to the 3-HB region. SEQ ID NO: 15, refers to amino acids 741-841 of TcdB of C. difficile corresponding to the beta flap region. SEQ ID NO: 16, refers to amino acids 1-541 of TcdA of C. difficile. SEQ ID NO: 17, refers to amino acids 1073-1452 of TcdA of C. difficile. SEQ ID NO: 18, refers to amino acids 22-62 of TcdA of C. difficile. SEQ ID NO: 19, refers to amino acids 146-536 of TcdA of C. difficile. SEQ ID NO: 20, refers to amino acids 1789-1840 of TcdA of C. difficile. SEQ ID NO: 21, refers to amino acids 664-842 of TcdA of C. difficile. SEQ ID NO: 22, refers to amino acids 743-842 of TcdA of C. difficile.

In some embodiments, the hinge epitope may be targeted. As used herein, the hinge epitope comprises one, two, or all three of: the hinge (aa 1792-1834), the 3-HB (aa 766-841), and the β-flap (aa 742-765). These three structural units are separated in amino acid sequence but cluster together in 3D.

In some embodiment, the isolated polypeptide comprises a peptide that is at least 50% identical to the sequence thereof. In some embodiment, the isolated polypeptides comprise a peptide that is at least 60% identical to the sequence thereof. In some embodiment, the isolated polypeptide comprises a peptide that is at least 75% identical to the sequence thereof. In some embodiment, the isolated polypeptide comprises a peptide that is at least 90% identical to the sequence thereof. In some embodiment, the isolated polypeptide comprises a peptide that is at least 98% identical to the sequence thereof.

The present invention also features an immunogen comprising at least one polypeptide according to the present invention. In some embodiments, the immunogen is a divalent immunogen specific for two polypeptides according to the present invention. In some embodiments, the two polypeptides are mixed. In some embodiments, the two polypeptides are covalently bound. In some embodiments, the immunogen is a trivalent immunogen specific for three polypeptides according to the present invention. In some embodiments, the three polypeptides are mixed. In some embodiments, the two or three polypeptides are covalently bound. In some embodiments, the immunogen is a tetravalent immunogen specific for four polypeptides according to the present invention. In some embodiments, the four polypeptides are mixed. In some embodiments, the two, three, or four polypeptides are covalently bound.

In some embodiment, the present invention features a method of neutralizing the primary holotoxins of C. difficile. In some embodiment the method comprises of producing an immunogen of a holotoxin of C. difficile, and introducing the immunogen to a host so as to elicit an immune response to the immunogen, wherein the host produces an antibody specific for the holotoxin based on the immunogen.

As used herein an “immunogen” may refer to any compound that can elicit an immune response in a host. Non-limiting examples of an immunogen may include a binding agent, antigen-binding regions (V_{H}) of heavy-chain only antibodies, termed VHHs or nanobodies, antibodies, antibody fragments, small molecules or drugs. Any other appropriate immunogens by be considered. As used herein, a “host” may refer to a mammal such as, but not limited to, a mouse or a human.

In some embodiment, the present invention features a method of designing and producing a vaccine specific for a holotoxin of C. difficile. In some embodiment, the vaccine may comprise an immunogen of, but not limited to, any of the sequences listed above in Table 2. In certain embodiments the vaccine comprises an immunogen or vaccine similar to the sequences listed above in Table 2, e.g., a truncated version, an enlarged version, or one that is homologous. The present invention provides the first mouse CDI vaccine using the pore-B epitope (SEQ ID NO: 3). The present invention is not limited to mouse vaccines and includes vaccines for others such as humans.

The present invention also describes formulating antigens with novel Toll-like receptor (TLR) tri-agonist adjuvant platforms, which uses combinatorial chemistry to link three different TLR agonists together to form one adjuvant complex. The immunomodulatory activity of panels of TLR tri-agonist adjuvants can be evaluated to find whether they elicit unique antigen-specific immune responses, e.g., in vitro and/or in vivo. The top candidates may be evaluated to help generate effective vaccines.

The present invention also describes strategies for vaccine design and production. For example, the present invention describes a vaccine antigen (Ag) capture and in vivo delivery platform using an optimized microsphere capture system. Tags or other chemical cross-linkers may be used to attach the antigen to microspheres. For example, His-tagged proteins are expressed from plasmids containing the sequence of antigens using an in vitro transcription translation (IVTT) system or in vivo (E. coli). Streptavidin-coated microspheres may be conjugated with tris-NTA biotin linkers and then used to capture proteins expressed in E. coli or from IVTT reactions. The resulting Ag-conjugated microspheres are administered directly with or without TLR-agonist adjuvants to monitor the dynamics and isotypes of the antibody release. Ag was coated at a density of approximately 200,000 per bead. Immunogenicity studies revealed robust and durable Ag-specific responses. This shows the isolation of specific proteins from a complex mixture by conjugation onto microspheres and direct immunogenicity testing can be performed in a high-throughput and scalable fashion. The present invention is not limited to this particular method, and the present invention is not limited to His-tags.

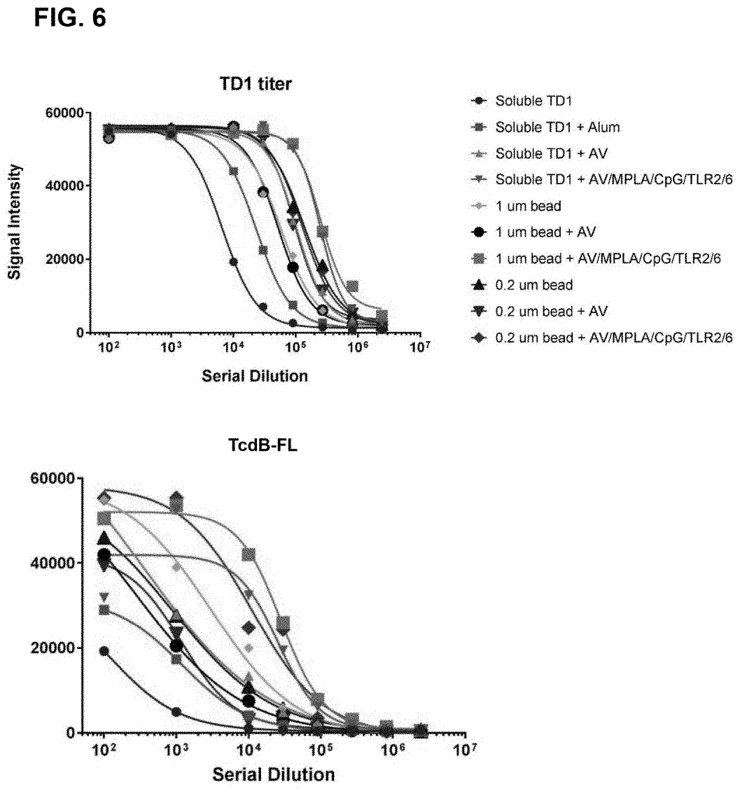

Vaccine formulations were produced according to Table 3. Mice were injected (SC) with the various formulations. Prime was Day 0; Boost 1 was Day 14, and there were 4 mice per group. Table 3 and FIG. 6 show measured midpoint titers. Ag stands for antigen alone; AV stands for Addavax; AV+TLR stands for Addavax, CpG, MPLA, TLR2,6. FIG. 6 shows antibody titers. Immunization with a non-toxic segment of C. difficile TcdB induces high antibody levels in mice. Antibody levels are boosted by greater than 3 logs. The immune response is specific against a 381 aa immunogen (compared to the full-length toxin, which is 2367 aa). The induced antibodies against the 381 aa immunogen also react to the full length toxin.

TABLE 3
Midpoint titer (serum dilution)

| Vaccine formulations | TD1 | TcdB-FL (full length) |
|---|---|---|
| Soluble TD1 | 6,888 | 43 |
| Soluble TD1 + Alum | 23,468 | 141 |
| Soluble TD1 + AV | 101,987 | 1,334 |
| Soluble TD1 + AV/MPLA/CpG/TLR2/6 | 237,546 | 13,323 |
| 1 uM Bead | 56,325 | 3,559 |
| 1 uM Bead + AV | 51,819 | 490 |
| 1 uM Bead + AV/MPLA/CpG TLR2/6 | 262,557 | 25,095 |
| 0.2 uM Bead | 137,560 | 1,015 |
| 0.2 uM Bead + AV | 102,618 | 469 |
| 0.2 uM Bead + AV/MPLA/CpG/TLR2/6 | 126,405 | 12,215 |

The present invention also describes methods for improving antitoxin activities of antibodies or binding agents and methods for developing multidomain antibodies or binding agents that simultaneously target multiple epitopes of interest (e.g., multiple neutralizing epitopes on the toxins herein).

The present invention describes targeting the neutralizing epitopes for inactivating TcdB for the treatment of CDI (e.g., with a drug, small molecule, binding agent, etc.). The present invention also describes the development of vaccines based on the neutralizing epitopes. An immunogen or vaccine can inactivate the holotoxin, e.g., by inhibiting the biological functions of individual domains that are prerequisite for its toxicity, or by promoting extracellular activation leading to its inactivation before it attacks cells.

### Example

The following is a non-limiting example of the present invention. It is to be understood that said example is not intended to limit the present invention in any way. Equivalents or substitutes are within the scope of the present invention.

### Methods

TcdB produced by the M68 strain of C. difficile was used. TcdB holotoxin and its GTD were expressed as described previously. The genes encoding the four VHHs (5D, E3, 7F, and B39), the GTD of TcdB produced by the VPI 10463 strain (residues 1-542, termed GTD^{VPI10463}), and a truncated Delivery/RBD of TcdB (residues 1072-1433, TcdB¹⁰⁷²⁻¹⁴³³), and TD1 (residues 1072-1452 with a 10×His-tag at the C-terminus) were cloned into a modified pET28a vector, which has a 6×His/SUMO (Saccharomyces cerevisiae Smt3p) tag introduced to the N-terminus of all proteins. A TcdB fragment (residues 1-1805, TcdB¹⁻¹⁸⁰⁵) was cloned into a modified pET22b vector, which has a twin-Strep tag introduced between the SUMO tag and TcdB¹⁻¹⁸⁰⁵ and a C-terminal 6×His tag. All mutants were generated by two-step PCR and verified by DNA sequencing.

5D, E3, 7F, B39, GTD^{VPI10463}, TcdB¹⁻¹⁸⁰⁵, TcdB¹⁰⁷²⁻¹⁴³³, and TD1 were expressed in Escherichia coli strain BL21-Star (DE3) (Invitrogen). Bacteria were cultured at 37° C. in LB medium containing kanamycin or ampicillin. The temperature was reduced to 16° C. when OD₆₀₀ reached ˜0.8. Expression was induced with 1 mM IPTG (isopropyl-b-D-thiogalactopyranoside) and continued at 16° C. overnight. The cells were harvested by centrifugation and stored at −80° C. until use.

The His₆-tagged TcdB, GTD, and the His₆-SUMO-tagged 5D, E3, 7F, B39, GTD^{VPI10463}, TcdB¹⁻¹⁸⁰⁵, TcdB¹⁰⁷²⁻¹⁴³³, and TD1 were purified using Ni²⁺-NTA (nitrilotriacetic acid, Qiagen) affinity resins in a buffer containing 50 mM Tris, pH 8.5, 400 mM NaCl, and 10 mM imidazole. The proteins were eluted with a high-imidazole buffer (50 mM Tris, pH 8.5, 400 mM NaCl, and 300 mM imidazole) and then dialyzed at 4° C. against a buffer containing 20 mM Tris, pH 8.5, 1 mM TCEP, and 40 mM NaCl. The His₆-SUMO tag of 5D, E3, 7F, B39, GTD^{VPI10463}, TcdB¹⁰⁷²⁻¹⁴³³, and TD1 were cleaved by SUMO protease. These proteins, as well as TcdB holotoxin and GTD with un-cleaved His-tag, were further purified by MonoQ ion-exchange chromatography (GE Healthcare) in a buffer containing 20 mM Tris, pH 8.5, and eluted with a NaCl gradient. TcdB¹⁻¹⁸⁰⁵, after cleaved by SUMO protease, was further purified using streptavidin resins.

The TcdB-5D-E3-7F complex was assembled by mixing the purified TcdB holotoxin with the 3 purified VHHs at a molar ratio of 1:2:2:2 for 2 hours on ice. The complex was then purified by MonoQ ion-exchange chromatography in 20 mM Tris, pH 8.5, followed by a Superose 6 size-exclusion chromatography (SEC; GE Healthcare) in 20 mM Tris, pH 8.5, 1 mM TCEP, and 40 mM NaCl. The GTD-E3, GTD^{VPI10463}-7F, TcdB¹⁰⁷²⁻¹⁴³³-5D complexes were made by mixing the purified GTD, GTD^{VPI10463}, and TcdB¹⁰⁷²⁻¹⁴³³ with E3, 7F, and 5D at a molar ratio of 1:2, respectively, for 2 hours on ice, followed by further purification using a MonoQ ion-exchange column (20 mM Tris, pH 8.5) and a Superdex-200 Increase SEC (20 mM Tris, pH 8.5, 1 mM TCEP, and 40 mM NaCl). All protein complexes were concentrated to ˜10 mg/ml and stored at −80° C. until use.

Tandem online Size-Exclusion Chromatography coupled to Small-Angle X-ray Scattering (SEC-SAXS) experiments were performed at SSRL beamline 4-2 as described previously. Purified TcdB holotoxin was exchanged into a buffer containing phosphate-buffered saline (PBS), pH 7.4, and 5 mM DTT, or 20 mM sodium acetate, pH 5.0, 50 mM NaCl, and 5 mM DTT, and then concentrated to 20 mg/ml. SEC-SAXS data were collected at pH 5.0 and 7.4 using Superdex-200 Increase PC 3.2/300 columns (GE Healthcare).

For DSSO cross-linking of TcdB, TcdB holotoxin (50 μL, 10 μM) in PBS buffer (pH 7.4) was reacted with DSSO at the molar ratio of 1:100 for 1 hr at room temperature. Cross-linking reaction was quenched by addition of 50-fold excess ammonium bicarbonate for 10 minutes, and the resulting products were subjected to enzymatic digestion using a FASP protocol. Briefly, cross-linked proteins were transferred into Milipore Microcon™ Ultracel PL-30 (30 kDa filters), reduced/alkylated and digested with Lys-C/trypsin sequentially as previously described. The resulting digests were desalted and fractionated by peptide SEC. The fractions containing cross-linked peptides were collected for subsequent MSⁿ analysis. In this work, three biological replicates were performed.

LC MSⁿ analysis was performed using a Thermo Scientific™ Dionex UltiMate 3000 system online coupled with an Orbitrap Fusion Lumos™ mass spectrometer. A 50 cm×75 μm Acclaim™ PepMap™ C18 column was used to separate peptides over a gradient of 1% to 25% ACN in 82 mins at a flow rate of 300 nL/min. Two different types of acquisition methods were utilized to maximize the identification of DSSO cross-linked peptides.

For single-molecule FRET analysis of TcdB, VHH-7F and B39 each contain a buried disulfide bond that renders the native cysteines inaccessible for labeling. A cysteine residue was introduced by mutagenesis into the N-terminus of 7F (at the −1 position) or into a surface-exposed loop in B39 (G42C). Expression and purification of the mutant VHHs were similar to the wild type proteins, except that 5 mM DTT was used in all the buffers during purification. The purified 7F was labeled with acceptor dye (Alexa-647 maleimide) while B39 was labeled with donor dye (Alexa-555 maleimide) (Thermo Fisher Scientific). The labeling efficiency was determined by UV-Vis spectroscopy to be >90%. The purified 5D was biotinylated using EZ-Link NHS-PEG4-Biotin (Thermo Fisher Scientific) at pH 6.8 to preferentially label the N-terminal amine TcdB holotoxin in complex with the Alexa-647-labeled 7F, the Alexa-555-labeled B39, and the biotin-labeled 5D was further purified using a Superose 6 SEC to remove the excess VHHs.

Cleaned quartz slides were passivated with biotinylated Bovine Serum Albumin followed by a mixture of 2% Biolipidure 203 and 0.2% Biolipidure 206 (NOF America Corp.) before the addition of streptavidin. Following this treatment, preformed TcdB-3VHH complex showed no nonspecific binding to the slide at concentrations orders of magnitude higher than the 100 pM concentrations used to achieve optical resolution between single molecules.

At such low protein concentrations, the non-covalently bound VHHs partially dissociated so measurements had to be made rapidly, which required seven repeated surface preparations at each pH condition. Samples were imaged using a prism-based Total Internal Reflection Fluorescence microscope. Samples were excited with a laser diode at 637 nm (Coherent Inc., Santa Clara, Calif.) for Alexa-647 and a diode pumped solid-state laser at 532 nm (Laser Quantum USA. Fremont, Calif.) for Alexa-555. Emission from donor and acceptor was separated using an Optosplit ratiometric image splitter (Cairn Research Ltd, Faversham UK) containing a 645 nm dichroic mirror with a 585/70 band pass filter for the donor channel and a 670/30 band pass filter for the acceptor channel (IDEX Health & Science. Rochester, N.Y.). The replicate images were relayed to a single iXon DU-897 EMCCD camera (Andor Technologies, Belfast, UK) at a frame rate of 10 Hz.

Data was processed in home written MATLAB scripts to cross-correlate the replicate images and extract time traces for diffraction limited spots with intensity above baseline. From the traces of fluorescence intensity over time for individual complexes, only those complexes containing a single donor and acceptor dye that showed anti-correlated photobleaching to baseline in a single time step were selected. From the magnitude of the anticorrelated photobleaching event, one can perform per-molecule γ-normalization, which allows us to report the absolute FRET efficiency. The FRET efficiency was compiled into histograms, which were fit to Gaussian functions.

To ensure that FRET changes were not the result of photophysical changes, the relative quantum yield and fluorescence anisotropy was measured for the free dyes, the dye-labeled VHHs, and the individual dye-labeled VHHs in complex with TcdB. All measurements were carried out at a dye concentration of 10 nM using the same buffers as the smFRET at pH 7 (50 mM Hepes, 100 mM NaCl, pH 7) and pH 5 (50 mM sodium acetate, 100 mM NaCl, pH 5).

Ensemble fluorescence was recorded on an ISS PC1 photon counting spectrofluorometer using a 2.0 mm excitation slit and a 2.0 mm emission slit. Alexa-555 and Alexa-647 labeled samples were excited at 532 nm at 637 nm respectively. Concentrations of samples used for fluorescence were determined from absorption measurements using the same cuvette. The emission intensity was taken as the sum of a 20 nm window about the emission maxima. Relative quantum yields were calculated by normalizing the intensities to the emission of free dye at pH 7. Anisotropy measurements were collected with 2.0 mm excitation slit and a 2.0 mm emission slit. Emission was recorded at 567 nm and 670 nm for the donor and acceptor, respectively. All measurements were done in triplicate and reported as the mean and standard error.

Dynamic light scattering (DLS) was carried out using a Zetasizer Nano S (Malvern Panalytical). TcdB was assayed at a concentration of 0.2 mg/ml in PBS buffer in a 200 μl volume cuvette at room temperature. Data were analyzed using Zetasizer Version 7.13 software.

For the calcein dye release assay, liposomes were prepared by extrusion method using Avanti Mini Extruder according to manufacturer's protocol. Briefly, lipids (Avanti Polar Lipid) at the indicated molar ratios were mixed in chloroform and then dried under nitrogen gas and placed under vacuum for overnight. The dried lipids were rehydrated and were subjected to five rounds of freezing and thawing cycles. Unilamellar vesicles were prepared by extrusion through a 200 nm pore membrane using an Avanti Mini Extruder according to the manufacturer's instructions.

Dried lipids containing 55% 1,2-dioleoyl-sn-glycero-3-phosphocholine (DOPC), 15% 1,2-dioleoyl-sn-glycero-3-phospho-L-serine (DOPS), and 30% cholesterol (10 mg/ml) were resuspended in 150 mM NaCl, 20 mM Hepes (pH 7.0), 1 mM EDTA, 50 mM calcein. Free calcein dye was separated from calcein-entrapped liposomes by desalting (Zeba). Fluorescence was measured on a Spectramax M2e cuvette module with excitation at 493 nm and emission at 525 nm. In the assay, liposomes were diluted in 150 mM NaCl, 20 mM sodium acetate (pH 4.6), 1 mM EDTA, to give a final concentration of 0.3 mM and incubated until the fluorescence signal was stable. TcdB alone (0-25 nM), or TcdB pre-incubated with 5D or 7F at a TcdB:VHH=1:2 molar ratio, was added and the fluorescence intensity was recorded for 7 minutes. The reaction was stopped by adding 0.1% Trion X-100. The percentage of fluorescence change was calculated as the ((F−Fᵢₙᵢₜᵢₐₗ) (F_{final}−Fᵢₙᵢₜᵢₐₗ)). The initial rate of calcein dye release was deduced from the slope of the linear part of the curve. The experiments were repeated three times independently.

Membrane depolarization was measured as previously described with some modifications. Briefly, liposomes composed of 55% DOPC, 15% DOPS, 30% cholesterol were prepared in 200 mM NaCl, 1 mM KCl, 10 mM Hepes (pH 7.0). To create a trans-positive membrane potential (+135 mV), liposomes were diluted in 200 mM KCl, 1 mM NaCl, 10 mM sodium acetate (pH 4.6) to give a final concentration of 0.1 mM. Membrane potential was monitored using 12 μM ANS. Valinomycin was added at time 0-second to give a final concentration of 30 nM. At 180-second, 100 nM TcdB holotoxin alone, or TcdB pre-incubated with 0.02-1 μM 5D or 1 μM 7F, was added and the fluorescence intensity at 490 nm was monitored for 7 minutes with excitation at 380 nm. The reaction was stopped by adding 2 μM gramicidin from Bacillus anerinolyticus (Sigma-Aldrich). The fluorescence change relative to the maximal change in the presence of gramicidin was calculated as the ((F−Fᵢₙᵢₜᵢₐₗ)/(F_{final}−Fᵢₙᵢₜᵢₐₗ)). The experiments were repeated three times independently.

The TcdB autoprocessing assays were performed in 25 μl of 20 mM Tris-HCl, pH 8.0, which contained 0.4 μM of TcdB holotoxin or TcdB¹⁻¹⁸⁰⁵, InsP6 at the indicated concentrations, with or without 7F (2 μM). The reaction mixtures were incubated at 37° C. for 1 h, and then boiled for 5 min in SDS sample buffer to quench the reaction. The samples were examined by 4-20% SDS-PAGE and the TcdB fragments were visualized by Coomassie blue staining.

### Crystal Structure of the Full Length TcdB

The full length TcdB holotoxin from the M68 strain of C. difficile was expressed in the well-validated Bacillus megaterium system and purified to high homogeneity. After extensive crystallization screening and optimization and testing a large number of crystals at the synchrotron, the best X-ray diffraction data were collected at 3.87 Å resolution on a crystal of a heterotetrameric complex composed of TcdB and three neutralizing VHHs (5D, E3, and 7F). The TcdB-VHH complex was crystallized at pH 5.2, which is a physiologically relevant pH in an endosome (FIG. 1A, FIG. 1B and Table 2). A complete structure of TcdB holotoxin was built except for two small regions (residues 944-949 and 1032-1047) that have no visible electron density due to high structural flexibility (FIG. 1B)

The crystal structure reveals that TcdB is composed of three major components. The GTD (residues 1-544) and CPD (residues 545-841) form the center piece involving extensive inter-domain interactions. The Delivery/RBD (residues 842-1834) forms an extended module, interacting with both the GTD and the CPD on one side and pointing away from GTD/CPD. The most prominent finding is the elongated CROPs domain (residues 1835-2367), which emerges from the junction of the CPD and the Delivery/RBD and stretches ˜130 Å in the opposite direction to curve around the GTD like a hook (FIG. 1B). The overall architecture of TcdB at endosomal pH is distinct from structural models of TcdB and TcdA that were derived from an EM study at neutral pH, where the CROPs lies in parallel to and interacts with the Delivery/RBD. Furthermore, the hydrophobic pore-forming region of TcdB (residues 957-1129 in the Delivery/RBD) was observed in a different conformation than that seen in a TcdA fragment near neutral pH. This likely represents a rarely seen pore-forming intermediate state of TcdB at endosomal pH, which is “frozen” by a neutralizing antibody (5D).

### The Unique Structure of the CROPs Domain

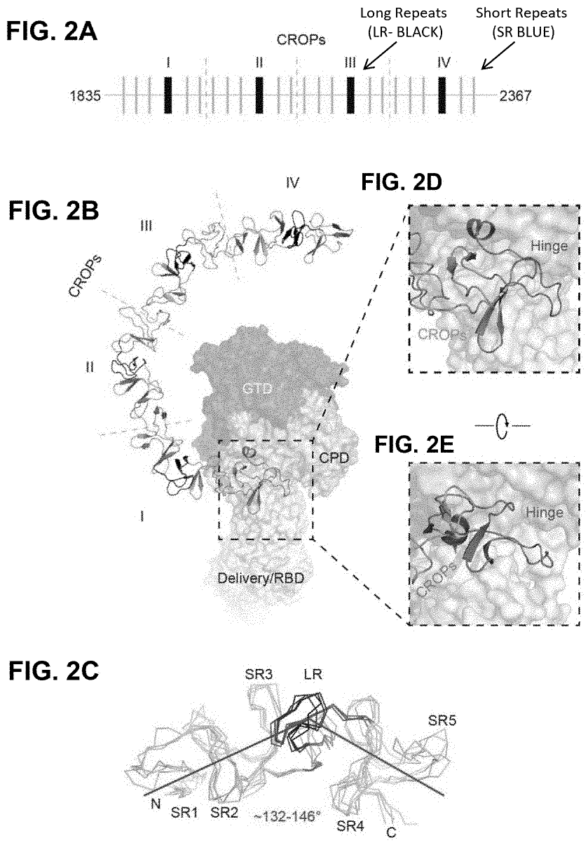

The CROPs of TcdB is composed of two types of repetitive sequences including twenty short repeats of 20-23 residues (termed SRs) and four long repeats of 30 residues (termed LRs) (FIG. 2A). Each SR consists of a β-hairpin followed by a flexible loop, while each LR has three β-strands that form a twisted anti-parallel β-sheet together with the β-hairpin of the preceding SR. The curvature of the CROPs arises because the straight, rod-like segments of the β-solenoid composed of SRs are interrupted by the interspersed LRs, which cause a ˜132-146° kink (FIG. 2B, FIG. 2C). Structurally, the CROPs could be divided into four equivalent units (termed CROPs I-IV), each is composed of a SR1-SR2-SR3-LR-SR4-SR5 module (FIG. 2C). Superposition of CROPs I-IV yielded a Cα root-mean square deviation (r.m.s.d.) of ˜0.9-2.6 Å.

Interestingly, an unrecognized SR module (residues 1815-1834) was identified at the C-terminus of the Delivery/RBD, which is like all other SRs. This new SR, together with an upstream long loop and a short a helix, form a structurally distinct module (residues 1792-1834), which is referred to herein to as the “hinge” because it connects the Delivery/RBD to the elongated CROPs. Furthermore, the hinge directly interacts with a three-stranded β sheet in the CPD (residues 742-765, termed the β-flap) that is crucial for CPD activation, as well as a 3-helical bundle (residues 766-841, referred to as 3-HB) that is located in a crevice surrounded by GTD, CPD, Delivery/RBD, and CROPs (FIG. 2D, FIG. 2E). Because of its strategic location, this hinge is primed to mediate structural communications among all four domains of TcdB. A functional role for this hinge is supported by earlier studies showing that deletions in this area drastically reduced the toxicity. Additionally, hypervariable sequences near the hinge may contribute to differences in toxicity and antigenicity displayed by TcdB variants produced by the hypervirulent C. difficile 027 ribotype and other less virulent strains.

### TcdB Displays Distinct Structures at Neutral and Acidic pH

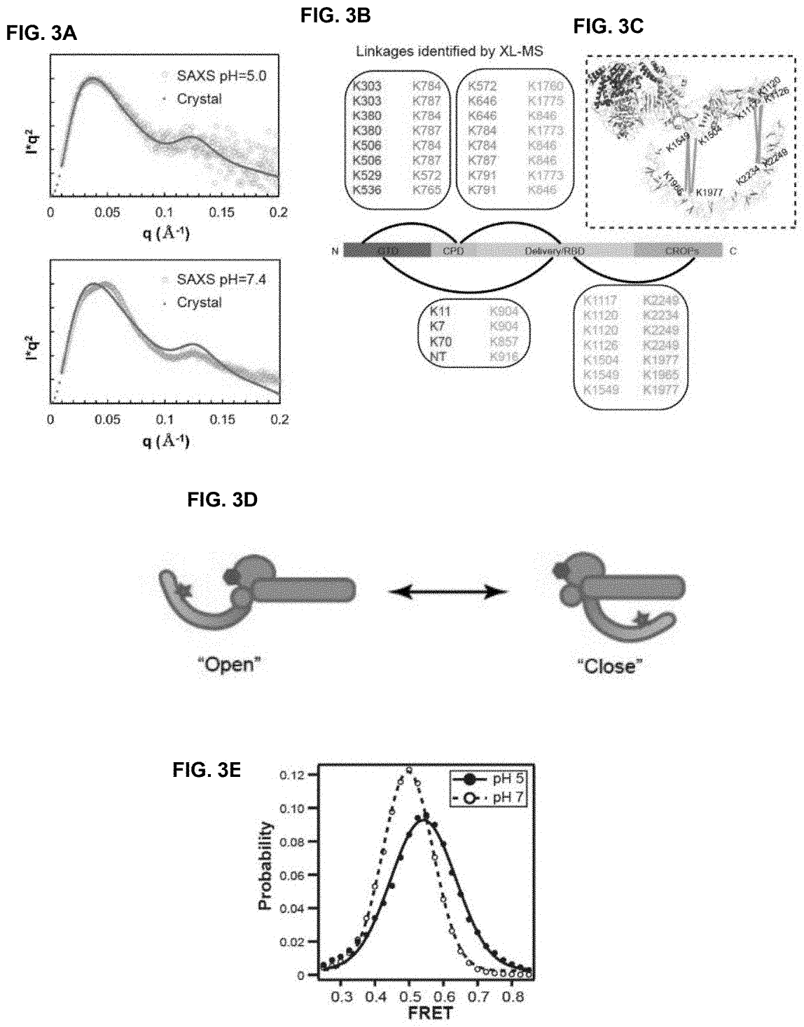

As the structure of TcdB holotoxin is derived from a crystal grown at an acidic pH, its solution structure was further examined using online size-exclusion chromatography coupled to SAXS (SEC-SAXS) at pH 5.0 and pH 7.4, respectively. Curve-fit analysis showed that the calculated scattering profile based on this crystal structure is nearly identical to the experimental scattering profile at pH 5.0, suggesting that the solution structure of TcdB is similar to the crystal structure at pH 5.0. However, disagreement at the middle-angle (middle q) region of the scattering profile between experimental SAXS data at pH 7.4 and the calculated profile for the crystal structure suggests that TcdB adopts a different conformation at neutral pH (FIG. 3A). Guinier and P(r) analyses showed similar R_{g} values at pH 5.0 and 7.4, however Dₘₐₓ of pH 5.0 (˜233.0 Å) was longer than that of pH 7.4 (˜205.0 Å). The Dₘₐₓ of TcdB at pH 5.0 is comparable to the value predicted from this crystal structure (˜247 Å). However, the shorter Dₘₐₓ of TcdB holotoxin at pH 7.4 is comparable to the value predicted from the TcdB core composed of the GTD, CPD, and Delivery/RBD (˜203 Å). It thus suggests that at pH 7.4 the elongated CROPs may swing towards the TcdB core to adopt a more compact conformation.

To better characterize the conformation of the CROPs at pH 7.4, XL-MS strategy was employed to determine inter-domain interactions of TcdB using DSSO (disuccinimidyl sulfoxide), a sulfoxide-containing MS-cleavable cross-linker. In total, 87 cross-links have been identified, representing 27 inter-domain and 60 intra-domain interactions in TcdB at pH 7.4. Among them, 8, 4, and 8 pairs of unique cross-linked peptides were identified between GTD and CPD, GTD and Delivery/RBD, and CPD and Delivery/RBD, respectively (FIG. 3B). When the XL-MS data was mapped to this crystal structure, almost all of these cross-links satisfy the distance cutoff of 30 Å, indicating a good correlation with the crystal structure of TcdB.

Interestingly, 7 pairs of cross-linked peptides were identified between the CROPs and the Delivery/RBD, which correspond to Cα-Cα distances ranging between 90 Å and 210 Å as measured in this crystal structure. This suggested that the CROPs of TcdB could move much closer to the Delivery/RBD at neutral pH than observed in this crystal structure. Specifically, the central portion of the CROPs around residues K1965 and K1977 and the C-terminal tip of the CROPs around residues K2234 and K2249 must be able to move within ˜30 Å of the Delivery/RBD (FIG. 3C). This new conformation would be consistent with the Dₘₐₓ of TcdB at pH 7.4 that was derived from SAXS studies, and similar to the “closed” conformation of TcdA at neutral pH. Since XL-MS enables the capture of dynamic and transient contacts in addition to stable structures, the time that TcdB spends in a “closed” TcdA-like conformation at neutral pH remains unknown.

pH-Dependent Structural Flexibility of the CROPs

Next smFRET was used to probe the pH-dependent conformational change of the CROPs. smFRET is a well-established method to probe protein structure and conformational changes, which can identify individual species in heterogeneous or dynamic mixtures. As TcdB has nine cysteine residues and C699 is crucial for the CPD function, three VHHs (7F, B39, and 5D) were used as molecular tools to label and capture TcdB rather than chemically label the toxin. Specifically, the acceptor dye (Alexa-647) was attached to a cysteine residue introduced at the −1 position of 7F, which labels the core of TcdB holotoxin. The donor dye (Alexa-555) was attached to B39, which specifically binds to the CROPs IV (PDB code: 4NC2). Given the structure of TcdB holotoxin, the distance between the two dyes is ˜47 Å. Energy transfer between these two dye-labeled VHHs monitors the movement of the CROPs (FIG. 3D). Biotin-labeled 5D, which has no effect on TcdB conformational change based on an ensemble FRET study, was used for immuno-pulldown of TcdB onto a passivated quartz microscope slide. The three VHHs were preassembled with TcdB and the complex was purified by size-exclusion chromatography.

From the traces of fluorescence intensity over time for individual heterotetrameric TcdB-VHH complexes, only those complexes containing a single donor and acceptor dye that showed anti-correlated photobleaching to baseline in a single time step were selected. Using the magnitude of the anticorrelated photobleaching event, per-molecule γ-normalization was performed, which allows us to report the absolute FRET efficiency. The FRET efficiency was compiled into histograms, which revealed single FRET peaks at both pH 5.0 and 7.0 (FIG. 3E). The presence of a single peak would be consistent with a static structure or dynamic averaging faster than the time binning of 100 ms, which cannot be distinguished by a single FRET pair.

A statistically significant difference was observed in the mean FRET efficiencies at pH 5.0 (0.532±0.015) and pH 7.0 (0.484±0.007), supporting the notion that TcdB displays a pH-dependent conformational change (FIG. 3E). A simple calculation from the mean FRET efficiency between the dye-labeled VHHs at pH 5.0 gives an estimated distance of 49.9±0.05 Å, which is consistent with the crystal structure of TcdB holotoxin at acidic pH (˜47 Å). Similar results were observed at pH 5.5 and pH 5.25. At pH 7.0, the mean FRET efficiency suggested the distance between labeling sites increases to 51.5±0.05 Å. A single FRET pair is insufficient to position the CROPs relative to the rest of TcdB, and any change in conformational dynamics would affect the simple conversion of FRET to distance. This slight increase in apparent mean FRET was accompanied by a statistically-significant 25% decrease in distribution width at pH 7.0 (0.113±0.002) relative to pH 5.0 (0.141±0.026), which is consistent with an increase in the rate of conformational dynamics.

Thus far, two limiting structural states have been identified in TcdB: an “open” conformation at acidic pH that is supported by the crystal structure, SAXS, and smFRET studies and a “closed” conformation at neutral pH revealed by SAXS and XL-MS studies (FIG. 3D). These data collectively suggest that the CROPs likely samples an ensemble of conformations relative to the core of TcdB at neutral pH, and such protein dynamics would not be resolved by the 100 ms integration time in smFRET. The lack of stabilizing contacts between the CROPs and the TcdB core and a potential structural rearrangement in the hinge that connects the Delivery/RBD and the CROPs should permit such conformational sampling.

### A Pore Forming Intermediate State of TcdB at Endosomal pH

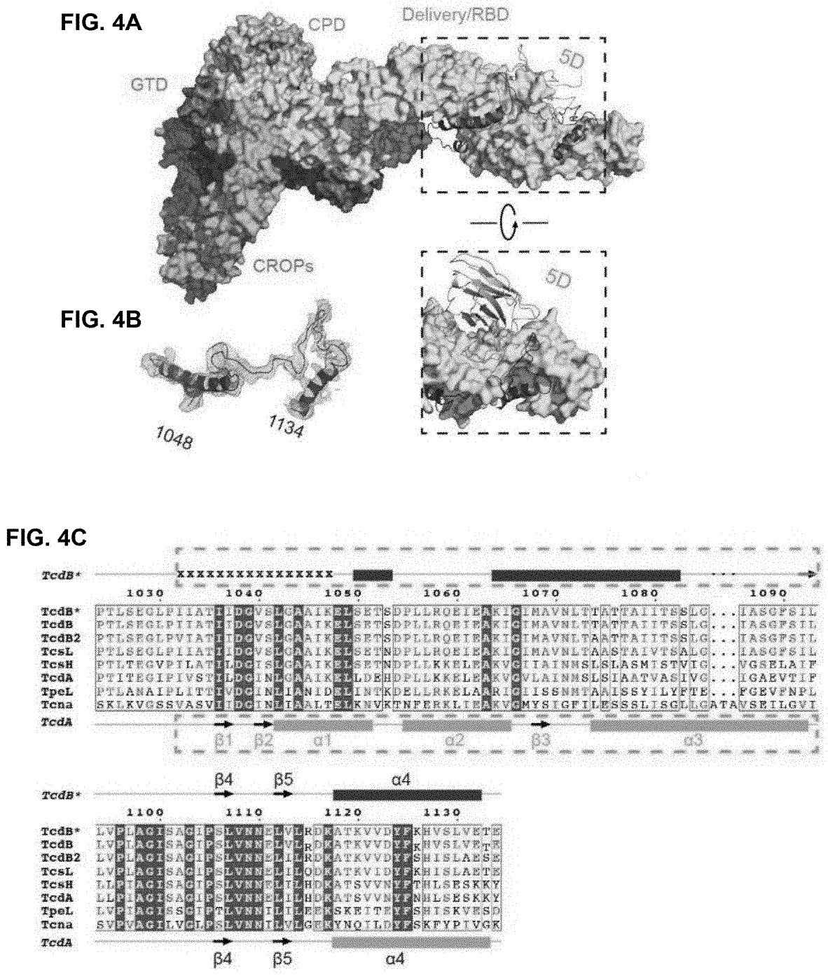

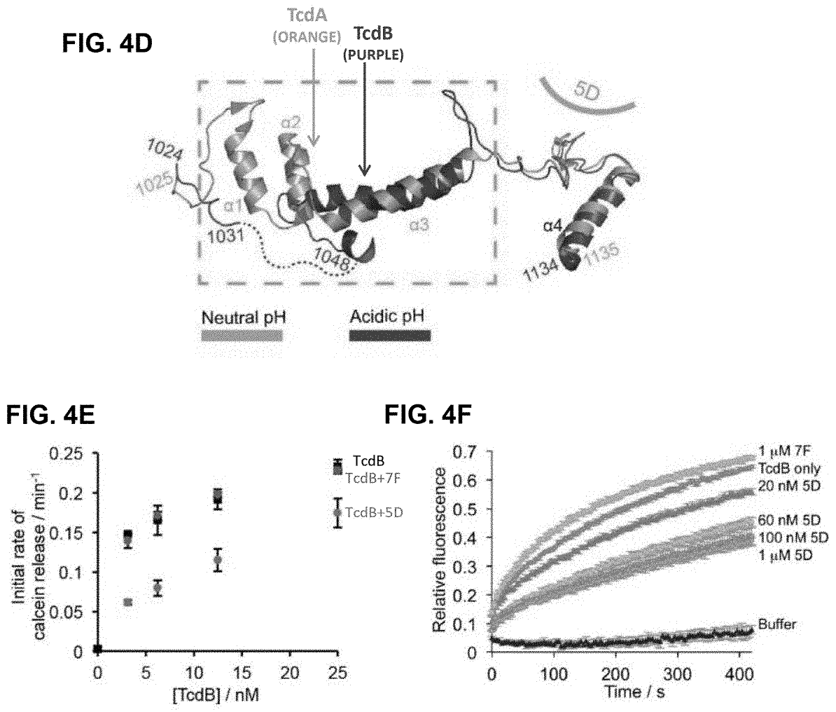

The Delivery/RBD serves to protect the hydrophobic pore-forming region (residues 957-1129), which is predicted to be released upon endosome acidification in order to form a pore that delivers the GTD and the CPD to the cytosol. The pore forming activity of TcdB also contributes to cell necrosis observed in vitro. A structural comparison between TcdB holotoxin at acidic pH and a TcdA fragment at neutral pH reveals drastic differences in the homologous C-terminal portion of the pore-forming region (residues 1032-1134 in TcdB and 1033-1135 in TcdA) (FIG. 4A, FIG. 4B). In TcdA, this region adopts a mixed α/β configuration, where hydrophobic residues are shielded in a continuous groove formed mostly by β-sheets in the Delivery/RBD (FIG. 4C, FIG. 4D). However, in the acidic conformation of TcdB, there was no electron density visible for residues 1032 to 1047, likely due to high flexibility, indicating that these residues unfolded and detached from the toxin core at endosomal pH. Furthermore, TcdB residues equivalent to the α2 in TcdA unfolded into a loop, while TcdB residues equivalent to the β3 and part of the α3 in TcdA assembled into a new helix that occupied the same area as the original α3 in TcdA. Because of this transition, hydrophobic residues in TcdB (residues 1084-1094) that are equivalent to the C-terminal portion of the α3 in TcdA bulged out as an extended loop. Intriguingly, the conformational change did not spread into the region where TcdB is bound by 5D, which maintains a similar conformation as that observed in TcdA.

To further dissect the contributions of acidic pH and 5D to the observed conformational changes in the pore-forming region, the crystal structure of a fragment of the Delivery/RBD, TcdB^{1072-1433 in} complex with 5D at pH 8.5 was determined (Table 2). It was found that the pore-forming region observed in TcdB¹⁰⁷²⁻¹⁴³³ at pH 8.5 adopts a TcdA-like neutral pH conformation. This finding thus suggests that the novel conformation in the pore-forming region observed in TcdB holotoxin likely represents an intermediate state induced by endosomal pH.

Furthermore, it was found that the binding mode of 5D to TcdB is almost identical at pH 8.5 and 5.2, involving all three complementarity-determining regions (CDRs) of 5D. The overall binding affinity of 5D is further strengthened by extensive polar and hydrophobic interactions involving TcdB residues outside the pore-forming region. Therefore, 5D can fix the conformation of β4-β5 in TcdB, which would prevent the pH-induced conformational changes in the β4-β5-α4 module. Prior mutagenesis studies showed that mutations introduced around the 5D-binding site in TcdB effectively inhibited pore formation and cellular toxicity, and mutating L1107 alone (L1107K) that is targeted by 5D caused a >1,000-fold decreased toxicity. These findings suggest that 5D likely inhibits the conformational changes necessary for pore formation by TcdB at endosomal pH.

To test this hypothesis, how 5D effects membrane insertion of TcdB was examined using two complementary assays. By monitoring the ability of TcdB to permeabilize calcein-entrapped liposomes, it was found that TcdB increased the rate of calcein release at pH 4.6 in a protein concentration dependent fashion (FIG. 4E). The rate of TcdB-induced dye release was significantly reduced when TcdB was pre-incubated with 5D. As a control, 7F, which binds the GTD, showed no effect on TcdB-induced dye release. The influence of 5D or 7F on TcdB to dissipate valinomycin-induced membrane potential in liposomes was further studied, and it was found that 5D, not 7F, reduced the ability of TcdB to depolarize membrane (FIG. 4F).

Taken together, these findings suggest that 5D neutralizes TcdB by preventing the pore-forming region from completing the necessary pH-induced conformational change. Notably, the pore-forming region recognized by 5D are highly conserved among a family of large clostridial glucosylating toxins (LCGTs), which include TcdA and TcdB, C. novyi α-toxin (Tcnα), C. sordellii lethal and hemorrhagic toxins (TcsL and TcsH), and C. perfringens toxin (TpeL) (FIG. 4C). Therefore, this portion of the pore-forming region represents a good target for the development of broad-spectrum vaccines and antibodies targeting TcdA, TcdB, other LCGTs, or other appropriate targets.

### Modulation of Autoprocessing of TcdB

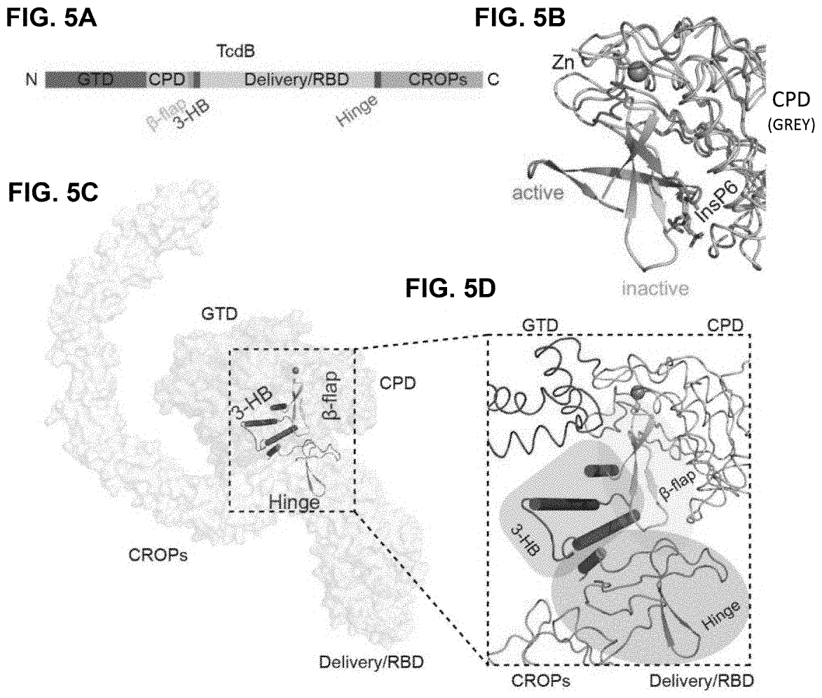

Activation of the CPD by InsP6 upon cell entry is a critical step in regulating the pathology of TcdA and TcdB. Overall, the structures of the apo-CPD in TcdB holotoxin and an InsP6-bound CPD fragment (PDB: 3PEE) are very similar (r.m.s.d. of ˜1.1 Å) except for the β-flap (FIG. 5A, FIG. 5B). The structure of the apo-CPD was compared with structures of a CPD fragment bound to InsP6 or a peptide inhibitor based on the cleavage sequence of TcdB (G⁵⁴²SL⁵⁴⁴) (PDB: 3PA8), and it was found that the β-flap partially occupies the P1 substrate pocket of the CPD in TcdB holotoxin, which would prevent substrate binding. In the CPD fragment, InsP6 triggers a ˜90° rotation of the β-flap (FIG. 5B), which activates the CPD by properly ordering the active site and the substrate pocket. However, such a rotation of the β-flap is prohibited in TcdB holotoxin, because it would otherwise sterically clash with the 3-HB that follows (FIG. 5D).

Besides allosteric modulation by InsP6, some studies suggested that the CROPs also affects TcdB autoprocessing. The efficiency of InsP6-induced GTD cleavage was compared using TcdB holotoxin and a truncated TcdB without the hinge and the CROPs (residues 1-1805). It was found that the InsP6-induced cleavage of the GTD was much more efficient in TcdB¹⁻¹⁸⁰⁵, suggesting that the CROPs and the hinge helps to inhibit the CPD function in TcdB holotoxin. Furthermore, in the absence of the CROPs, a TcdB fragment that carries the hinge (residues 1-1832) showed a weaker InsP6-dependent cleavage of GTD than the one without the hinge (residues 1-1795). These data suggest that the hinge is involved in regulation of TcdB autoprocessing. Notably, in TcdB holotoxin, the hinge interacts with the β-flap and the 3-HB, together forming the “heart” of TcdB that connects all four domains (FIG. 5C, FIG. 5D). Since the β-flap and the 3-HB are important for coupling between InsP6 binding and CPD activation, structural rearrangement in the hinge, associated with pH-dependent movement of the CROPs, could contribute to the regulation of CPD function.

### VHH 7F and E3 Reveal Two Distinct Neutralizing Epitopes on the GTD

7F inhibits GTD cleavage, but does not directly interact with the CPD. Instead, 7F binds to the C-terminus of the GTD, immediately juxtaposed to the cleavage site (L544). Notably, the CDR3 of 7F binds to an α helix (residues 525-539) upstream of the scissile bond, as well as a neighboring α helix (residues 137-158) with extensive polar and hydrophobic interactions. Such interactions interfere with the movement of the scissile bond into the CPD cleavage site and a proper orientation of GTD relative to CPD, and thus inhibiting cleavage of the GTD.

E3 inhibits Rho glucosylation and blocks the cytopathic effects of TcdB by specifically targeting the GTD. In two independently solved crystal structures using the GTD fragment or TcdB holotoxin, E3 binds to the N-terminal four-helix bundle (residues 1-90) in a similar manner. More specifically, E3 recognizes the 2^{nd} and the 3^{rd} helixes (residues 21-64) in the GTD with extensive polar and hydrophobic interactions. Since structure of a GTD-Rho complex has not been reported, it remains unknown how E3 may affect GTD-Rho interactions or the catalysis. The homologous four-helix bundle is also found in the glucosyltransferase domain of other LCGT members, which may be involved in plasma membrane binding of the glucosyltransferase domain, suggesting that E3 may interfere with membrane association of the GTD. The structure of the GTD-E3 complex thus lays the foundation for further validating and exploiting of these mechanisms as a new strategy to counteract TcdB and potentially other LCGT members.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

1.-31. (canceled)

32. An isolated immunogenic polypeptide that is a fragment of a TcdB holotoxin or a TcdA holotoxin of Clostridium difficile.

33. The polypeptide of claim 32, wherein the polypeptide is selected from a group consisting of: SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, and SEQ ID NO: 22.

34. The polypeptide of claim 32, wherein the polypeptide is at least 75%, 90%, or 98% identical to SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

35. The polypeptide of claim 32, wherein the fragment is smaller than a whole holotoxin protein but larger than a 15-mer peptide.

36. An immunogen comprising at least one polypeptide according to claim 32.

37. The immunogen of claim 36, wherein the polypeptide is selected from a group consisting of: SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, and SEQ ID NO: 22.

38. The immunogen of claim 36, wherein the polypeptide is at least 75%, 90%, or 98% identical to SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

39. A vaccine comprising an immunogen having at least one polypeptide according to claim 32.

40. The vaccine of 39, wherein the polypeptide is selected from a group consisting of: SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, and SEQ ID NO: 22.

41. The vaccine of claim 39, wherein the polypeptide is at least 75%, 90%, or 98% identical to SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

42. The vaccine of claim 39, wherein the vaccine is specific for a holotoxin C. difficile

43. The vaccine of claim 39, wherein the vaccine is a human vaccine.

44. A method of neutralizing a holotoxin of C. difficile, the method comprising producing an immunogen of a holotoxin of C. difficile, and introducing the immunogen to a host so as to elicit an immune response to the immunogen, wherein the host produces an antibody specific for the holotoxin based on the immunogen.

45. The method of claim 44, wherein the holotoxin is TcdB or TcdA.

46. The method of claim of 44, wherein the immunogen comprises a polypeptide sequence that is selected from a group consisting of: SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, and SEQ ID NO: 22.

47. The method of claim 44, wherein the immunogen comprises a polypeptide sequence that is at least 75%, 90%, or 98% identical to SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

48. A method of designing and producing a vaccine specific for a holotoxin C. difficile the method comprising:
a) expressing a tagged protein from a plasmid containing the sequence for an immunogen; and
b) capturing the tagged protein on a microsphere.

49. The method of claim 48, wherein the holotoxin is TcdB or TcdA.

50. The method of claim of 48, wherein the immunogen comprises a polypeptide sequence that is selected from a group consisting of: SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, and SEQ ID NO: 22.

51. The method of claim 48, wherein the immunogen comprises a polypeptide sequence that is at least 75%, 90%, or 98% identical to SEQ ID NO: 3, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.
